(19) United States
(12) Patent Application Publication
(10) Pub. No.: US 2014/0080214 A1
(43) Pub. Date: Mar. 20, 2014

(54) CLEAVABLE CELLULOSIC SPONGE DEVELOPMENT FOR 3 DIMENSIONAL CELL CULTURE AND SPHEROIDS RETRIEVAL

(71) Applicant: Agency for Science, Technology and Research, Connexis (SG)

(72) Inventors: Bramasta Nugraha, Singapore (SG); Hanry Yu, Singapore (SG)

(21) Appl. No.: 13/737,812

(22) Filed: Jan. 9, 2013

(30) Foreign Application Priority Data
Sep. 20, 2012 (SG) 201207005-8

(51) Int. Cl. C12N 5/00

(52) U.S. Cl. CPC C12N 5/0062
USPC 435/395; 536/92

(57) ABSTRACT

A method of making a scaffold for 3 dimensional cell culture comprising the steps of conjugating a reducible disulfide bond onto a hydroxyl group at the side chain of a hydroxypropyl cellulose; forming a matrix of hydroxypropyl cellulose having the reducible disulfide bond conjugated onto the hydroxyl group such that a reducible disulfide bond exists adjacent to a double bond for crosslinking the matrix of hydroxypropyl cellulose. The scaffold and a system of using the scaffold for culturing 3 dimensional cell spheroids

### RELATED APPLICATIONS

This application claims priority to Singaporean Patent Application Serial No. 201207005-8, filed Sep. 20, 2012, incorporated herein by reference.

### FIELD

The invention relates to scaffolds for cell culture and methods of making and using the same.

### BACKGROUND

In tissue engineering scaffold design, it is preferable to have the scaffold that is biodegradable because three dimensional scaffolds serves only as temporary support for cell growth [1]. Upon study completion or for further downstream cell assays, the bulk amount of the scaffold materials need not be present hence biodegradability is important. Ideally, scaffold degradation products should not elicit cytotoxicity or immunological responses and it is desired to have water soluble degradation product for easy removal of by products. As shown previously, cellulosic sponge has shown many salient features for cell culture applications such as high water uptake, excellent hydrophilicity, diffusible and aqueous macroporosity, versatility for ligand conjugation, soft mechanical stiffness suitable for soft tissue culture, induces quick hepatocyte repolarization through accelerated spheroids formation, ease of cell seeding procedure and capability for large scale cell culture and drug/anti viral screenings [2-4].

Macroporous hydrogel sponge made of cellulose derivative could entrap cells, induce spheroids formation and maintain these constructs within the macroporous networks over extended culture period. However the current cellulosic sponge design is physically stable during cell culture thus imposing challenges to retrieval of the spheroids from the sponge for further analysis and further use. Addition of trypsin or other dissociating enzymes to retrieve these spheroids is unable to achieve good spheroids harvesting yield. Biodegradation capability of this class of cellulosic sponge has not been investigated. The reason is probably due to its rare application as a tissue engineering scaffold.

To our knowledge, a few others have explored a way to degrade cellulose derivative materials using cellulase enzyme from Aspergillus niger to hydrolyze 1,4-β-D-glycosidic linkages but it might not be suitable for cell culture applications involving sensitive cells e.g. primary hepatocyte and stem cells [5]. Our attempt to use this enzyme has also proven that the enzyme did not cleave the rather strong covalent bonds of the cellulosic sponge. Other class of polymeric scaffold such as polyester has been explored as hydrolysis induced-degradable scaffold but the degradation mechanism is rather slow and sometimes unpredictable [6]. Although the degradation occurs physiologically, they only exhibit gradual degradation kinetics with degradation times ranging from days to months [7]. In addition, this continuous hydrolysis process leads to the gradual weakening of the system during tissue growth. It is desirable to maintain the scaffold mechanical stiffness to support the cell/tissue growth throughout the culture that still allows on demand degradation kinetics upon completion of the study. The possibility of attaching UV-sensitive bond in the scaffold chemical construct is an option to degrade the scaffold on demand, but the use of high power and intensity of UV to cleave the labile bond might be detrimental to mature and sensitive cells in culture [8]. Disulfide bonds have been used as cleavable bonds for various applications ranging from drug delivery to tissue engineering researches [9, 10]. In particular for soft tissue culture, disulfide-containing scaffold was found to be suitable in 3D environment of in vitro tissue culture to be utilized for replacing diseased tissue in vivo [7].

Disulfide bond cleavability has been known for years in protein synthesis mechanism. When cysteine is oxidized it forms cystine, which is actually two cysteine residues joined by a disulfide bond (cysteine-S—S-cysteine) between the —SH group [11]. In recent years, there is an increasing interest to prepare scaffold matrices containing disulfide crosslinked bonds since cleaving these bonds is controllable under physiological condition by altering the concentration of reductant used [12-15]. Through chemical cleaving, disulfide bond (—S—S—) would readily decompose into thiol groups (—SH HS—). These thiol groups do not decrease the local pH as is commonly observed in polyester hydrolysis mechanism [16]. Various chemical reductants are known to cleave disulfide bonds e.g. dithiothreitol (DTT), glutathione (GSH), L-cysteine (Cys) and tris(2-carboxyethyl)phosphine (TCEP) [13, 17-20]. The insertion of this disulfide bond into the hydrogel construct has been known to induce cleavage in physiological condition by incubating with reductants. Soluble decomposed products were easily removed by washing with excess cell culture medium or buffer [21]. Various disulfide-containing hydrogels were already reported as temporary hydrogel template, ECM-mimicry matrix and 3D cell encapsulation platform [1, 13, 19, 21]; however there is no exploration on cleavable macroporous disulfide-containing cellulosic sponge as hydrogel template for spheroids culture and retrieval. All the known reductants would either damage spheroids culture or need to be used at such high concentrations that would damage sensitive primary cells grown in spheroid cultures.

Many types of mammalian cells can aggregate and differentiate into 3-Dimensional multicellular spheroids when cultured in suspension or a nonadhesive environment. Compared to conventional monolayer cultures, multicellular spheroids better resemble real tissues in terms of structure and function. Multicellular spheroids may be used for tumor models and for therapeutic screening. Many primary or progenitor cells show significantly enhanced viability and functional performance when grown as spheroids. Multicellular spheroids in this aspect are ideal building units for tissue reconstruction.

An object of the invention is to ameliorate at least one of the above mentioned problems.

### SUMMARY

We have designed a cellulosic-sponge scaffold and method of making a cellulosic sponge scaffold that may be quickly cleavable

Accordingly a first aspect of the invention includes a method of making a scaffold for 3 dimensional cell culture comprising the steps of conjugating a reducible disulfide bond onto a hydroxyl group at the side chain of a hydroxypropyl cellulose; forming a matrix of the hydroxypropyl cellulose having the reducible disulfide bond conjugated onto the hydroxyl group such that the reducible disulfide bond exists adjacent to a double bond for crosslinking the matrix of hydroxypropyl cellulose.

Another aspect of the of the invention is includes a scaffold for 3 dimensional cell culture is also described comprising a reducible disulfide bond conjugated onto a hydroxyl group at the side chain of a hydroxypropyl cellulose adjacent to a double bond for crosslinking the matrix of the hydroxypropyl cellulose.

Another aspect of the of the invention is includes a system of culturing 3 dimensional cell spheroids comprising, seeding cells on a scaffold of the invention; and incubating the cells in a cell growth medium allowing the cells to grow into a spheroid.

The disulphide bonds may provide the advantage of allowing scaffold cleavage at physiological conditions within a short amount of time.

Cleavage of the disulphide bonds may provide the advantage of reducing cell toxicity, minimizing disruption of tight cell-cell junction and maintaining primary cell polarity. This may result in retrieval of viable cultured cells in higher numbers than is currently available.

Other aspects and advantages of the invention will become apparent to those skilled in the art from a review of the ensuing description, which proceeds with reference to the following illustrative drawings of preferred embodiments.

### BRIEF DESCRIPTION OF THE DRAWINGS

FIG. 1. Schematic diagram of cleavable cellulosic sponge synthesis and fabrication.

FIG. 2. Cleavable sponge chemical structure validation by ¹H NMR.

FIG. 3. N1s XPS analysis of conjugated galactose indicates the increase of net N1s counts after conjugation.

FIG. 4. Sponge physical characteristics: a) Sponge top view, b) SEM images of the sponge surface porosity (insert image is the surface nanoroughness view at higher magnification (scale bar 1 μm), c) Sponge aqueous macroporosity.

FIG. 5. Sponge cleavage characterizations: FTIR analysis indicates the presence of valley at 2550 cm⁻¹ wave number.

FIG. 6. Sponge cleavage characterizations: Ellman's thiol analysis reveals the ability of thiol groups from the cleaved HPCSS sponge to further cleave (5,5′-dithiobis-(2-nitrobenzoic acid) thus capture UV absorbance at 412 nm.

FIG. 7. Sponge cleavage characterizations: Physical morphology changes of HPCSS Gal sponge upon addition of tris(2-carboxyethyl)phosphine (TCEP) and dithiothreitol (DTT) at various concentrations (in mM unit).

FIG. 8. Dynamic observation of sponge cleavage with 10 mM TCEP. Aqueous macroporosity disappears after 30 minutes incubation of 10 mM TCEP (scale bar 200 μm).

FIG. 9. Schematic drawing of cleavable cellulosic sponge cleavage mechanism

FIG. 10. TCEP toxicity study on rat hepatocyte indicates good maintenance of cell viability (>80%) (scale bar 100 μm).

FIG. 11. Rat hepatocytes cultured in cleavable HPCSS Gal sponge. Compact spheroids are formed within one day post-seeding (scale bar 100 μm). Average spheroids size on day 1: 75.58±16.61 μm, day 3: 84.68±17.64 μm, day 5: 96.95±13.52 μm and day 7: 97.88±11.84 μm.

FIG. 12. Live/dead staining of rat hepatocyte spheroids in the cleavable sponge (scale bar 20 μm).

FIG. 13. Retrieved rat hepatocyte yield percentage upon sponge cleavage.

FIG. 14. Gene expressions analysis of rat hepatocyte cultured in cleavable HPCSS Gal sponge indicate no significant effect of the incubation of 10 mM TCEP towards CYP450 enzymes (***p value>0.06, ** p value>0.25, # p value>0.4, p value>0.5).

FIG. 15. Immunofluorescence staining of polarity markers and cell-cell junction of hepatocyte spheroids retrieved from cleavable HPCSS Gal sponge and cultured in non-cleavable HA Gal sponge. The incubation of 10 mM TCEP to cleave the sponge does not show harmful effect to these markers (scale bar 20 μm).

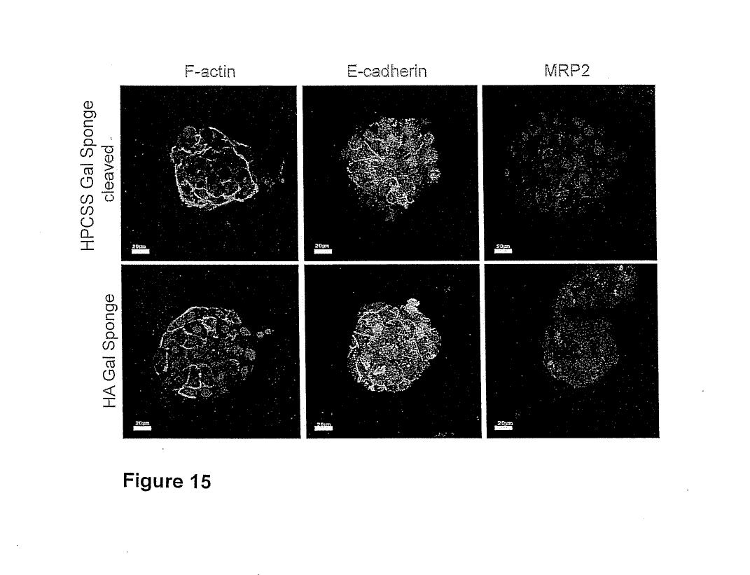

FIG. 16. FDA staining of the retrieved rat hepatocyte spheroids from cleavable HPCSS Gal sponge indicates putative accumulation of dye at the bile canaliculi region (scale bar 20 μm).

FIG. 17. Rat hepatocyte spheroids SEM images obtained from both cleavable HPCSS Gal sponge and non-cleavable HA Gal sponge indicate no surface morphology difference.

FIG. 18. Retrieved rat hepatocyte spheroids replated on collagen dish indicates the ability of the spheroids to spread on collagen (scale bar of zoomed out images 100 μm) (Insert is the zoomed in view of the spheroid, scale bar 20 μm).

FIG. 19. Retrieved hepatocyte spheroids can also be replated on poly-L-lysine dish to prevent spheroids spreading (top panel scale bar 100 μm, bottom panel scale bar 20 μm).

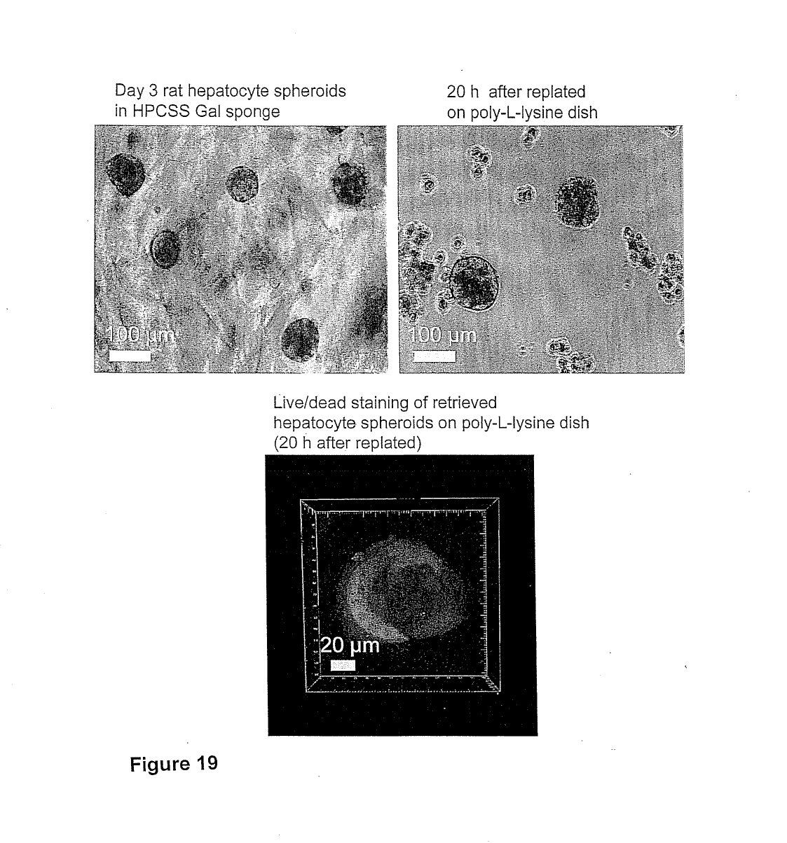

FIG. 20. E1 and mESCs cells cultured in HPCSS sponge: a) Phase contrast images, b) Spheroids retrieval and c) Live/dead staining.

### DETAILED DESCRIPTION OF THE PREFERRED EMBODIMENTS

A method of making a scaffold for 3 dimensional cell culture is described comprising the steps of conjugating a reducible disulfide bond onto a hydroxyl group at the side chain of a hydroxypropyl cellulose; forming a matrix of the hydroxypropyl cellulose having the reducible disulfide bond conjugated onto the hydroxyl group such that the reducible disulfide bond exists adjacent to a double bond for crosslinking the matrix of hydroxypropyl cellulose.

The location of the reducible bond may provide the advantage of a rapid degradation that will allow spheroids to be quickly recovered, thereby minimizing the cells exposure to any chemicals used to reduce the disulfide bond

Preferably, the matrix comprises a macroporous network.

A macroporus network may have the advantage of providing adequate space and support for the growth of spheroids.

Preferably, the method further comprises the step of conjugating a legend onto the matrix. Any ligand capable of conjugating to the hydroxyl groups of the cellulose matrix would be suitable. This may include, a galactose ligand or any ligands with functional groups that are able to conjugate with the cellulose sponge such as primary amines.

Preferably, the reducible disulfide bond comprises a dithiodipropionic acid.

Preferably, the double bond crosslinking the matrix of hydroxypropyl cellulose is crosslinked by gamma radiation.

Preferably, the reducible disulfide bond is reducible by a reductant. The reductant may include dithiothreitol (DTT), glutathione (GSH), L-cysteine (Cys) and tris(2-carboxyethyl)phosphine (TCEP) or any other reductant known in the art to reduce disulphide bonds.

In a preferred embodiment the reductant is tris(2-carboxyethyl)phosphine (TCEP).

This has the advantage of being less toxic to cells than other known reductants. The less toxicity on cells further reduces the cells exposure to toxicity that is already provided by the location of the disulphide bond that allows quick recovery of spheroids.

A scaffold for 3 dimensional cell culture is also described comprising a reducible disulfide bond conjugated onto a hydroxyl group at the side chain of a hydroxypropyl cellulose adjacent to a double bond for crosslinking the matrix of the hydroxypropyl cellulose.

Preferably, the matrix of the scaffold comprises a macroporous network

Preferably, the scaffold further comprising a ligand conjugated onto the matrix.

Preferably, the reducible disulfide bond of the scaffold comprises a dithiodipropionic acid.

Preferably, the reducible disulfide bond of the scaffold is reducible by a reductant. The reductant may include dithiothreitol (DTT), glutathione (GSH), L-cysteine (Cys) and tris(2-carboxyethyl)phosphine (TCEP) or any other reductant known in the art to reduce disulphide bonds.

In a preferred embodiment the reductant is tris(2-carboxyethyl)phosphine (TCEP).

A system of culturing 3 dimensional cell spheroids is also described comprising, seeding cells on a scaffold as described herein; and incubating the cells in a cell growth medium allowing the cells to grow into a spheroid.

Preferably, the system further comprises cleaving the disulfide bond with a reductant to retrieve the spheroid. The reductant may include dithiothreitol (DTT), glutathione (GSH), L-cysteine (Cys) and tris(2-carboxyethyl)phosphine (TCEP) or any other reductant known in the art to reduce disulphide bonds.

In a preferred embodiment the reductant is tris(2-carboxyethyl)phosphine (TCEP).

Preferably, the cells could be any cells able to grow and form spheroids.

In one embodiment the cells are hepatocytes.

In one embodiment the cells are metastatic cancer cells.

In one embodiment cells are pluripotent cells.

The disulfide-containing cellulosic sponge is designed to be used as 3D spheroids culture and retrieval of these spheroids. Cell types that show good functionality if cultured as spheroids are explored such as hepatocyte cells, cancer cells and stem cells. However, any cells able to form spheroids could be used.

With this respect, disulfide bonds are of our particular interest since they are stable against hydrolysis but can be cleaved on demand in the presence of reducing agents.

The choice of a less cytotoxic reductant was identified. Tris(2-carboxyethyl)phosphine (TCEP), a chemical reductant, was used to cleave the disulfide bonds in the cellulosic macroporous sponge. Compared to a known and strong reductant dithiothreitol (DTT), Surprisingly not only was TCEP found to be more stable, it was also found to be more effective in cleaving disulfide bonds and able to reduce disulfide bonds at wider pH range; TCEP is effective at pH 1.5-8.5, while DTT is only effective at pH 7-8.1. This has the advantage of allowing spheroid retrieval from 3-D cell culture at the natural culture pH.

In terms of cytotoxicity, although 10 mg/mL TCEP was found to reduce cell viability upon 24 hours of exposure to the cells it was still in the acceptable range of 60% remaining viable cells. In our attempt to develop disulfide-containing cellulosic sponge, we aim to synthesize a sponge which can be cleaved in less than 1 hour thus reducing the exposure time between the cells and the reductant, therefore careful polymer design is important.

Here, we have demonstrated by conjugating a reducible disulfide bond onto a hydroxyl group at the side chain of hydroxypropyl cellulose prior to attaching the double bond for crosslinking, we can tailor the cellulosic sponge to be readily cleavable on-demand upon addition of suitable disulphide bond reductant that works best under a range of cell-compatible conditions. The sponge cleavage occurs at physiological condition (pH 7.4, 37° C., in cell culture medium and or buffer) within 30 minutes without inducing significant cell toxicity, measured as disruption of cell-cell junction, primary hepatocyte polarity markers and/or cytochrome P450 enzymes. When cultured in our cleavable sponge, E1 cells (metastatic derivative human colorectal cancer cells) and mouse embryonic stem cells (mESCs) also exhibited compact spheroids formations with good cell viability during culture and upon retrieval. The insertion of a disulfide bond at the side chain of hydroxypropyl cellulose also does not interfere with the formation of macroporous network of cellulosic sponge or the crosslinkability of the hydrogel with y irradiation. Moreover this modification does not inhibit the ligand conjugation process onto the sponge for subsequent cell culture application.

Comparing our disulfide-containing cellulosic sponge to the other disulfide-containing hydrogel systems, our technology shows the importance of having macroporous networks with mechanically and chemically tunable properties of the sponge for 3D cell culture within diffusible construct rather than cell encapsulated in hydrogel system. The presence of macroporous networks is hypothesized to accelerate the sponge cleavage. Sponge macroporosity helps in the formation of cell-dense construct i.e. spheroids. Our disulfide-containing sponge also shows significantly faster cleavage rate at the addition of comparable reductants concentration than other disulfide-containing hydrogels (which cannot form spheroids as in our sponge likely due to blockage of cell-cell interactions by hydrogels) i.e. spheroids can be easily retrieved from our sponge as fast as 30 minutes. Compared with the prior art of cellulosic sponge [2], this cleavable disulfide-containing cellulosic sponge has been designed and synthesized to minimize cytotoxicity and maximize retrieval of viable cells. The reduction condition importance for cytocompatibility with rapid sponge cleavage for intended applications with utilities have been explored and experimentally determined.

The disulphide bonds are directly bonded to the hydroxyl groups at the side chain of the hydroxylpropyl cellulose of the cellulose sponge.

Comparing our disulfide-containing cellulosic sponge to the other disulfide-containing hydrogel systems, our technology shows the importance of having macroporous networks with mechanically and chemically tunable properties in the sponge for 3D cell culture within diffusible construct rather than cell encapsulated in hydrogel system. Our disulfide-containing sponge also shows faster cleavage rate at the addition of comparable reductants concentration than other disulfide-containing hydrogels (which cannot form spheroids as in our sponge possibly due to blockage of cell-cell interactions by hydrogels), i.e. spheroids can be easily retrieved from our sponge in as fast as 30 minutes.

One can theoretically develop a different material that has the similar macroporosity, mechanical and chemical tenability, cell biocompatibility, and sensitive/rapid reductant cleavability (with different types of biocompatible reductants), e.g. enzymes to cleave peptide that is sensitive to matrix-metaloproteases (Lutolf M P, et al. PNAS 100 (2003) 5413-5418) or UV-cleavable hydrogels (A. M. Kloxin, et al. Science 324 (2009) 59-63). However, the combinations of all these characteristics are required for the intended 3D cell culture with strong cell-cell interactions in epithelial tissues for in vitro and in vivo applications of tissue-engineered constructs.

Macroporous hydrogel sponge made of cellulose derivative could entrap cells, induce spheroids formation and maintain these constructs within the macroporous networks over extended culture period. However, the current cellulosic sponge design is physically stable during and after cell culture, thus limiting the retrieval of cells from the sponge for analysis and further use. Addition of trypsin or other dissociating enzymes to retrieve these spheroids is unable to achieve good harvesting yield.

By conjugating reducible disulphide bond into hydroxyl groups at the side chain of hydroxypropyl cellulose prior to attaching the double bond for crosslinking, we can tailor the cellulosic sponge to be readily cleavable upon addition of suitable disulphide bond reductant that works best under a range of cell-compatible conditions. The sponge cleavage occurs at physiological conditions (pH 7.4, 37° C., in cell culture medium and or buffer) within 30 minutes without inducing significant cell toxicity, disruption of tight cell-cell junction and primary cell polarity markers. The insertion of disulphide bond at the side chain of hydroxypropyl cellulose also does not interfere with the formation of macroporous network of cellulosic sponge, as well as the crosslinkability of the hydrogel with gamma irradiation. Moreover, this modification does not inhibit the ligand conjugation onto the sponge for subsequent cell culture application.

This material provides the capability of culturing various types of cells in three dimensions within the macroporous networks of cellulosic sponge, maintaining the cell culture for extended periods, and retrieving the cells for further downstream applications/assays. The ability of retrieving the cells from the sponge could ease the downstream functional analysis, which is normally challenged by the physical stability of other existing materials.

The scaffold preferably provides a platform to culture cells in three dimensional macroporous networks, as well as to safely retrieve the cell constructs formed therein.

The system demonstrates maintenance and growth of viability cells and cell polarity or cell-cell junction maintenance even in the presence of the reductant. In our case we have demonstrated 30 minutes sponge cleavage with non-toxic reductant concentrations and with cells in culture.

Materials

All chemicals and reagents were purchased from Sigma Aldrich (Singapore), unless otherwise stated.

Chemical Synthesis of Disulfide-Containing Hydroxypropyl Cellulose Polymer (HPCSS)

Hydroxypropyl cellulose (HPC) Mw=80,000 g/mol and degree of etherification ˜3.4 was dehydrated by azeotropic distillation in toluene at 70° C. 1 gram of dried HPC was dissolved in 30 mL anhydrous dimethylformamide (DMF), to which 5.95 mmol of 4-dimethylaminopyridine (DMAP) was added. In a separate flask, 5.95 mmol of dithiodipropionic acid (DTDP) was dissolved in 30 mL anhydrous DMF. Completely dissolved DTDP was activated with 2.98 mmol of each 1-ethyl-3-(3-dimethylaminopropyl)carbodiimide (EDCl) and N-hydroxy succinimide (NHS) for 10 minutes. The activated DTDP solution was added to HPC/DMAP mixture and reacted for 24 hours at room temperature under N₂ blanket. The mixture was then dialyzed in excess methanol and water, subsequently, and lyophilized. The end product is denoted as HPCDTP.

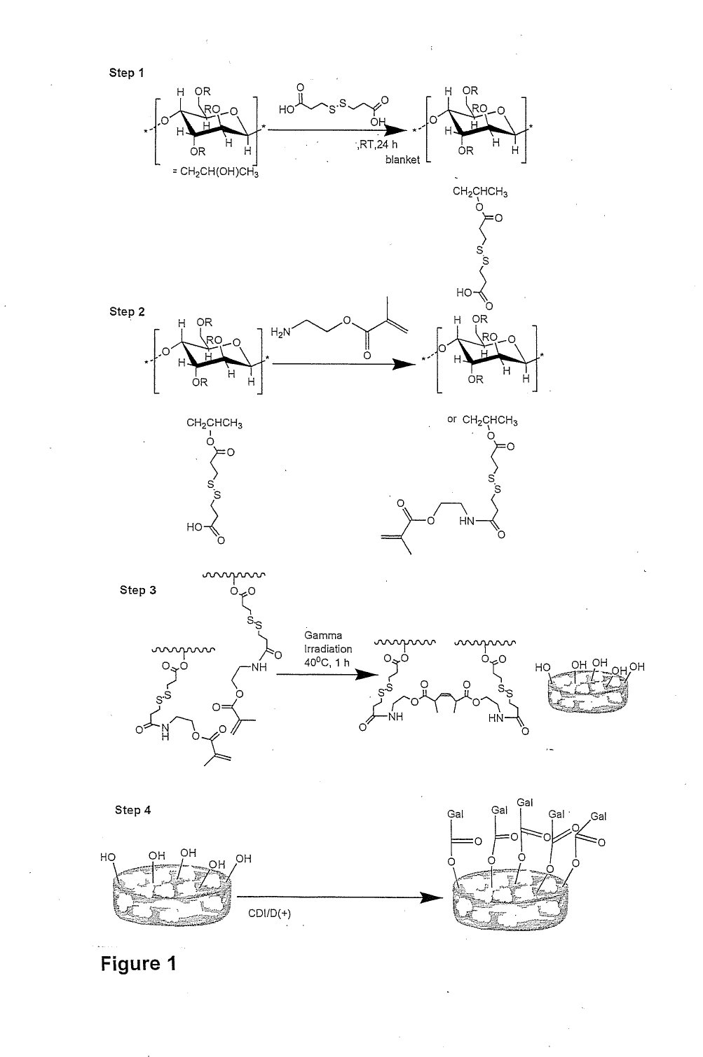

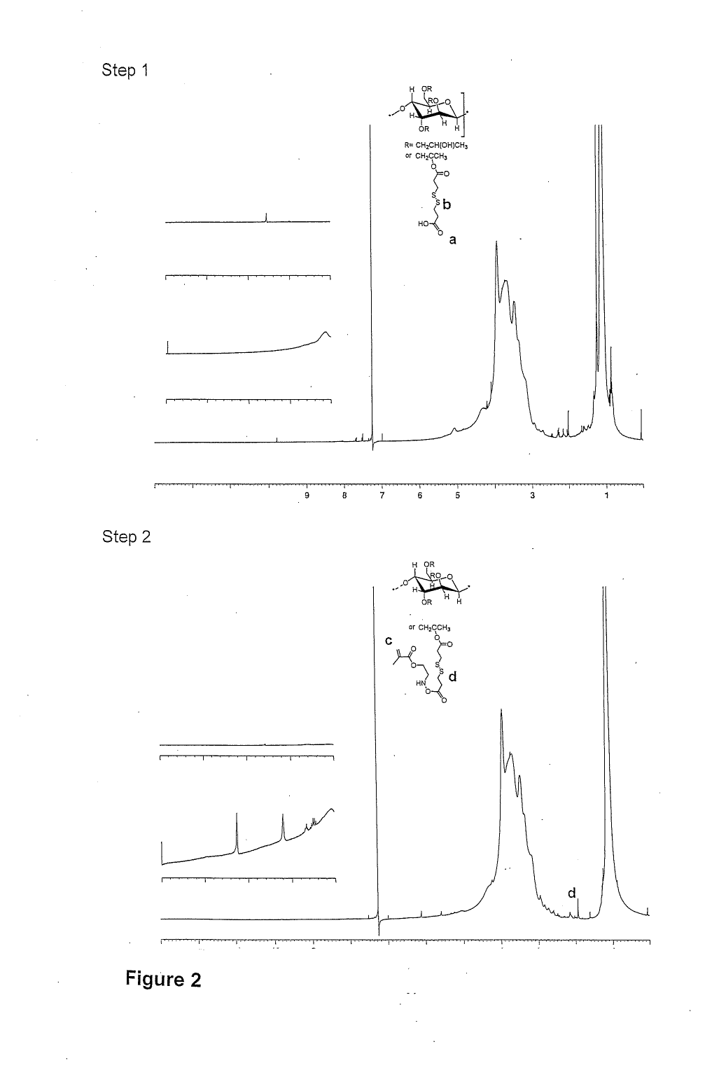

HPCDTP was dissolved in 30 mL deionized water and activated with two molar ratio of each EDCl and N-hydroxysulfosuccinimide (sulfo-NHS) for 10 minutes. In a separate flask, two molar ratio of 2-amino ethyl methacrylate hydrochloride was dissolved in 30 mL deionized water. 2-amino ethyl methacrylate hydrochloride solution was added to the activated HPCDTP mixture and reacted for 24 hours at room temperature under N₂ blanket. The mixture was then dialyzed in excess water subsequently and lyophilized. The end product is denoted as HPCSS. A schematic diagram with the complete synthesis described, including ¹H NMR characterization are shown in FIGS. 1 and 2.

Preparation of HPCSS Sponges

Firstly, HPCSS was dissolved in deionised water to a final concentration of 10% wt/vol after which the solution was inserted into glass tubes (diameter 10 mm, length 6 cm). The tubes were heated in a 40° C. water bath until phase separation occurred and then crosslinked by y irradiation for 1 hour at a dose of 10 kGray/hour (Gammacell 220, MDS Nordion, Canada). The sponge monoliths were obtained by breaking tubes subsequent to freezing the frozen glass tubes in dry ice. A Krumdieck tissue slicer (Alabama Research & Development USA) with set speed 50 rpm was used to cut the sponge uniformly. Sliced sponges were further washed extensively with excess amounts of deionised water for 3 days to remove uncross-linked polymers. These sponges were lyophilized prior to further galactosylation.

Galactosylation of HPCSS Sponges (HPCSS Gal Sponges)

The lyophilized pre-sliced HPCSS sponge was immersed in acetone three times within 20 minutes interval each (1 mL of acetone per 10 mm diameter 1 mm thickness sponge). The sponge was further activated with 2 mM 1,1′-carbonyldiimidazole (CDl) dissolved in acetone for 30 minutes and shaken at 4° C. The activated sponge was washed with acetone three times to remove the excess CDl. 2 mg/mL of D-(+)-galactosamine dissolved in carbonate bicarbonate buffer pH 10 was added and reacted at 4° C. for 24 hours. The reacted sponge was subsequently washed with excess of Dulbecco's Phosphate Buffered Saline (DPBS) and deionized water three times each. Upon washing, sponge was lyophilized. The cleavability of the sponge was confirmed by further incubation of the sponge in 10 mM tris(2-carboxyethyl)phosphine (TCEP) in cell culture medium adjusted to pH 7.4.

Physiochemical Characterization of HPCSS Gal Macroporous Sponges

X-Ray Photoelectron Spectroscopy

X-Ray photoelectron spectroscopy was used to qualitatively verify galactose ligand conjugation onto the HPCSS sponge. Measurements were made on a VG ESCALAB Mk II spectrometer with a MgKa X-ray source (1253.6 eV photons) at a constant retard ratio of 40.

Elastic Modulus Measurement

The elastic modulus of the sponge was measured by atomic force microscopy (Bioscope Catalyst, Veeco Instruments, Santa Barbara, Calif.) in deionized water. A hybrid Atomic Force Microscopy (AFM) probe consisting of a silicon nitride cantilever and a silicon tip (ScanAsyst-Fluid, Veeco Probes, Camarillo, Calif.) was used. The deflection sensitivity was calibrated by ramping force-distance curves on a glass surface, and the spring constant was calibrated by the thermal noise method. After calibration, 128×128 force-distance curves were recorded over an area of 5 μm×5 μm by force volume. Each force-distance curve was analyzed by fitting to the Hertz model with conical tip geometry and Poisson ratio of 0.5. The obtained elastic moduli from each force-distance curve were mapped into a bitmap image with 128×128 pixels. The curve fitting and statistical analysis was implemented by a self-developed Fortran program. The relationship between elastic modulus with the measured force is described as

$$F = \frac{2}{π} \frac{E}{1 - {v}^{2}} tan α {δ}^{2} ,$$

where F is the measured force, E is Young's elastic modulus, v is the Poisson ratio of the material under measurement (0.5 was used in the data processing), α is the half angle of the probe (22°) and δ is the sample deformation.

Scanning Electron Microscopy

Top and cross section views of the sponge surface morphology and porosity were captured using SEM (JEOL JSM-5600, Japan) at 10 kV. High magnification of SEM (15,000 folds) was performed to observe the sponge surface nanostructure. Prior to imaging, the dried sponge was sputter coated with platinum for 90 seconds. Pore size distribution of the sponges was quantified with imageJ software (version 1.43u) from collective SEM top view images of the sponges.

Fourier Transform Infrared Spectroscopy

Infrared spectra were recorded on a Perkin Elmer Spectrum 100 FTIR (Fourier transform infrared) spectrometer. The disulfide to thiol exchange upon sponge cleavage was identified at wavenumber 2550 cm^{-1 [}21].

Ellman's Thiol Analysis

Crosslinked HPCSS gel was completely cleaved with 25 mM DTT in DPBS at room temperature for 90 minutes and was further dialyzed using a Spectra/Pore membrane (molecular weight cutoff 12-14,000) for 3 days to remove DTT. The dried decomposed product was obtained by lyophilizing the solution for 3 days. The decomposed product (0.15 mM) was dissolved in 2.4 mL of 0.1 M Tris-HCl/0.01 M EDTA buffer (pH=8.0) and 100 μL of 0.01 M 5,5′-dithio-bis(2-nitrobenzoic acid) (DTNB) (Sigma, USA)/0.05 M DPBS (pH=7.0) was added to the solution. The absorbance of the solution at 412 nm was measured by Agilent 8453 UV Visible System and readings were compared with 0.05 mM TCEP (as positive control), 0.15 mM cystamine (as negative control) and 0.15 mM uncleaved pre-crosslinked HPCSS polymer.

Sponge Cleavage Condition Optimization

For easy visualization during cleavage, HPCSS Gal sponges were pre-soaked in 0.25 mg/mL propidium iodide overnight (Molecular Probes, USA). The stained sponges were incubated with 3 mL each of TCEP and DTT (as known strong reductant) per sponge at various concentrations (25, 10, 5, 3 and 1 mM) in 37° C., 5% CO₂ and 95% incubator. The morphological changes of the sponge were monitored visually every 15 minutes.

Dynamic of Sponge Cleavage with Time Lapse Imaging

The propidium iodide-prestained HPCSS Gal sponges were incubated with 10 mM TCEP and its macroporosity changes were monitored by Olympus fluoview FV1000 equipped with 37° C. heated chamber with a 60× water lens for 30 minutes with 2 minutes time interval. Images were analysed using IMARIS and images assembled using Adobe illustrator CS3.

Cells Culture

Hepatocytes were isolated from male Wistar rats weighing 250-300 g using the in situ collagenase perfusion method [26]. Animals were handled according to the IACUC protocols approved by the IACUC committee of National University of Singapore. Viability of hepatocytes was determined to be >90% by the Trypan Blue exclusion assay. Yields were approximately 10⁸ cells/rat. Freshly isolated rat hepatocytes were seeded onto the sponge by simply dropping the cell suspension on the sponge surface (0.5×10⁶ cells per 55 μL cell suspension per 10 mm diameter 1 mm thick sponge). The cell suspension was slowly absorbed into the sponge interior due to the inherent hydrophilicity of the sponges. Fresh cell medium was added slowly to the sponge edge after 45 minutes incubation (500 μL per sponge in 24-well plate). Hepatocytes seeded on a collagen monolayer platform (0.29 mg/mL collagen concentration) were used as control. Cells were maintained with Williams' E medium supplemented with 10 mM NaHCO₃, 1 mg/mL BSA, 10 ng/mL of EGF, 0.5 mg/mL of insulin, 5 nM dexamethasone, 50 ng/mL linoleic acid, 100 units/mL penicillin, and 100 mg/mL streptomycin and were incubated with 5% CO₂ at 37° C. and 95% humidity. Medium was replenished every day.

E1 cells (metastatic derivative human colorectal cancer cells) and mouse embryonic stem cells (mESCs) were both obtained from American Type Culture Collection (Manassas, Va., USA). E1 cells were maintained in McCoy's 5A medium supplemented with 10% FBS, 100 U/mL penicillin, and 100 μg/mL streptomycin. mESCs were maintained in DMEM high glucose medium supplemented with 15% embryonic stem cell-grade FBS, 0.1 mM non essential amino acids, 2 mM L-glutamine, 1000 U/mL leukemia inhibitory factor (LIF), 0.1 mM β-mercaptoethanol, 100 U/mL penicillin, and 100 μg/mL streptomycin. E1 cells and mESCs were seeded onto the sponge by simply dropping the cell suspension on the sponge surface (0.3×10⁶ cells per 55 μL cell suspension per 10 mm diameter 1 mm thick sponge). The cell suspension was slowly absorbed into the sponge interior due to the inherent hydrophilicity of the sponges. Fresh cell medium was added slowly to the sponge edge after 45 minutes incubation (500 μL per sponge in 24-well plate). Mediums were replenished every 2 days.

TCEP Toxicity Study in Primary Rat Hepatocyte

Primary rat hepatocyte was seeded on collagen coated well in 24-well plate format (0.3×10⁶ cell per well). The culture was maintained for 3 days and the medium was replenished every day. On the 3^{rd} day, the cells were incubated with TCEP solution in Williams' E medium at pre-determined concentrations and time; 5 mM for 1.5 hour, 10 and 25 mM for 30 minutes each. The duration of the incubation was chosen based on the ability of the associated TCEP concentrations to completely cleave HPCSS Gal sponge. Upon incubation, the morphology of the cells was monitored and followed with further incubation of 1 mg/mL (3-(4,5-dimethylthiazol-2-yl)-2,5-diphenyltetrazolium bromide (MTT) solution for 3 hours. The formed formazan product in each well was dissolved with 300 μL of 0.04 M HCl in isopropanol and its optical density was measured by TECAM Infinite M1000 microplate reader at 570 nm. Cell viability was determined by calculating the ratio of treated sample absorbance against untreated sample absorbance.

Hepatocyte Spheroids Characterization and Functional Assessment

Spheroids Size Distribution

Spheroid size distribution was quantified using imageJ software (version 1.43 u) from collective phase contrast images of living hepatocyte spheroids cultured in the HPCSS Gal sponges on day 1, 3, 5 and 7.

Spheroids Retrieval by Cleaving the HPCSS Gal Sponge

Rat hepatocyte, E1 and mESCs spheroids were retrieved from the sponge by incubating cell seeded-cleavable sponge with 3 mL of 10 mM TCEP in respective cell culture mediums in each well of 12-well plate for 30 minutes at 37° C. (incubator set up 5% CO₂ and 95% humidity). The cleaved sponge solution from each well was mixed thoroughly by 3 mL plastic dropper and then transferred into 15 mL falcon tube. This solution was further diluted with 3 mL pre-warmed DPBS and centrifuged at 100×g for 10 minutes. This washing step was repeated three times to remove TCEP solution completely. Eventually, the retrieved hepatocyte spheroids were resuspended in fresh mediums and replated on collagen coated dish or poly-L-lysine coated dish for further assays or spheroids manipulation.

Live/Dead Staining

Spheroids were co-stained with Cell Tracker Green (CTG, 20 μM) (Molecular Probes, USA) and propidium iodide (PI, 25 μg/mL) (Molecular Probes, USA) to determine live and dead cells, respectively. Cells were incubated for 30 minutes at 37° C. and then fixed with 3.7% paraformaldehyde for 10 minutes at room temperature. Fluorsave (Merck Chemicals) was applied to the stained spheroids to minimize photo-bleaching. Images were acquired by confocal laser scanning microscopy (Zeiss LSM510, Germany) at 488 and 543 nm excitation wavelengths.

Reverse Transcriptase Polymerase Chain Reaction

RNA was extracted from hepatocytes cultured as 3D spheroids in HPCSS Gal sponges by RLT lysis buffer (Qiagen, Singapore). Total RNA concentration was quantified by a Nanodrop (Thermoscientific) and 1 μg of RNA was converted to cDNA by High Capacity RNA-to-cDNA (Applied Biosystems). Primers were designed using Primer 3 and real-time PCR was performed by using SYBR green fast master mix on a ABI 7500 Fast Real-Time PCR system (Applied Biosystems). Gene expression was calculated using the ΔΔCT method normalized to GAPDH. The primers used in experiment are shown below.

TABLE 6
Primer sequences used in RT-PCR experiments

| CYPs | | |
|---|---|---|
| Gene | Forward sequence | Reverse sequence |
| CYP1A2 | CACGGCTTTCTGACAGAC | CCAAGCCGAAGAGCATCACC |
| | CC (SEQ ID NO: 1) | (SEQ ID NO: 2) |
| CYP2B2 | ACCGGCTACCAACCCTTG | TGTGTGGTACTCCAATAGGG |
| | AT (SEQ ID NO: 3) | AACA (SEQ ID NO: 4) |
| CYP3A2 | TGGGACCCGCACACATGG | TCCGTGATGGCAAACAGAGG |
| | ACT (SEQ ID NO: 5) | CA(SEQ ID NO: 6) |

Immunofluorescence Microscopy

To stain F-actin, E-cadherin and MRP2, hepatocytes spheroids cultured for 72 hours post-seeding in the sponges and retrieved from HPCSS Gal sponges were fixed in 3.7% paraformaldehyde for 10 minutes. For staining F-actin, the cells were permeabilized for 5 minutes in 0.1% Triton X-100 and incubated with 1 μg/mL TRITC-phalloidin (Molecular Probes, USA) for 20 minutes. For E-cadherin and MRP2 staining, following washing and blocking with 2% BSA/0.2% Triton-X 100 the spheroids were incubated overnight at 4° C. with primary antibodies: anti-rat E-cadherin (BD, USA) and rabbit anti-rat MRP2 (Sigma Aldrich, Singapore), respectively. Secondary antibodies used were goat anti-mouse and goat anti-rabbit 488 and 555 (Molecular Probes, USA), respectively. Nuclei stain was captured using DAPI stain (Vecta Shield, UK). Images were captured using Olympus fluoview FV1000 with a 60× water lens. Images were analysed using IMARIS and assembled using Adobe illustrator CS3.

Biliary Excretion of Fluorescein Dye

For monitoring hepatocyte repolarization, we visualized the excretion of fluorescein dye via bile canaliculi. Hepatocytes spheroids retrieved from the cleaved sponge were incubated with 15 μg/mL fluorescein diacetate (Molecular Probes, USA) in Williams' E medium at 37° C. for 45 minutes. The cultures were then rinsed and viewed with a 63× water lens on a Zeiss Meta 510 confocal microscope.

Scanning Electron Microscopy

Hepatocyte spheroids retrieved from cleavable HPCSS Gal sponge and non cleavable HA Gal sponge control on day 3 were fixed with 3.7% paraformaldehyde overnight and stained with 1% OsO₄ for 1 hour. Samples were then dehydrated step-wise with ethanol (25%, 50%, 75%, 90% and 100%) for 10 minutes each, dried in a vacuum oven and sputter coated with platinum for 90 seconds. The samples were viewed with a scanning electron microscope (JEOL JSM-5600, Japan) at 10 kV.

Results

Versatility of Hydroxypropyl Cellulose Chemistry Facilitates Conjugation of Cleavable Disulfide Bonds

The first step in the chemical synthesis of the cleavable cellulosic sponges (HPCSS sponge) involved the conjugation of dithiodipropionic acid onto hydroxypropyl groups of hydroxypropyl cellulose to provide carboxylic acid group (—COOH) to further react with 2-amino ethyl methacrylate hydrochloride. The second step in the synthesis was the conjugation of 2-amino ethyl methacrylate hydrochloride onto carboxylic acid group. Conjugated methacrylate group acted as crosslinking site during y irradiation, similar function as the allyl group described previously [2]. The galactose conjugation onto the remaining available hydroxypropyl groups was performed using 1,1′-carbonyldiamidazole in acetone. The sponge chemical synthesis and fabrication are depicted in detail in FIG. 1.

Conjugated dithiodipropionic acid presence on the chemical backbone was verified by ¹H NMR by identifying singlet peak at ˜9.5 to 10 ppm (Step 1, FIG. 2). In estimation, there was at least 1 conjugated dithiodipropionic acid group in every 6 subunits of HPC. Upon further conjugation of 2-amino ethyl methacrylate hydrochloride in the step 2, this singlet peak disappeared. Based on calculation, the conjugated methacrylate group appeared in every 16 subunits of HPC. Multiplet peaks appeared at ˜5 to 5.5 ppm indicated successful conjugation of methacrylate group (Step 2, FIG. 2).

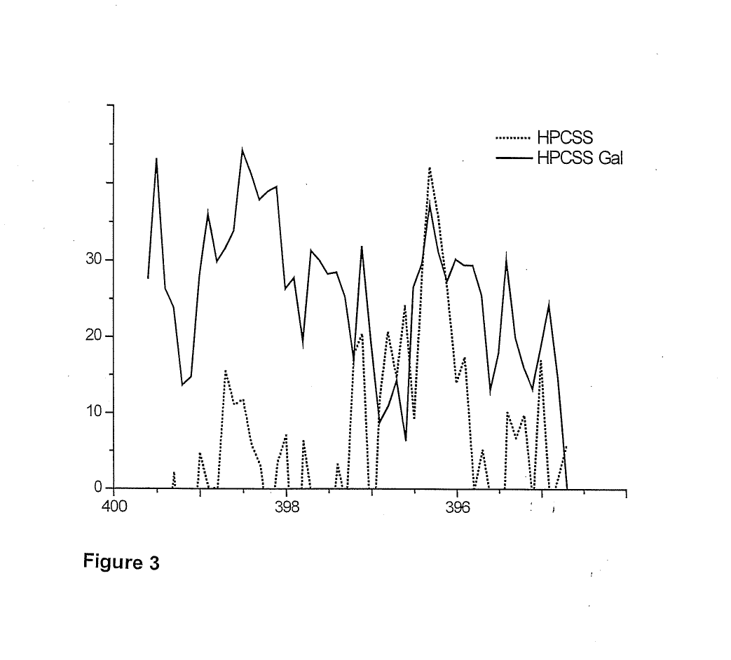

Galactose presence on the chemical backbone was quantified by N1s scan XPS. An XPS spectrum showed increased nitrogen atomic counts after conjugation (˜0.6% increase) (FIG. 3).

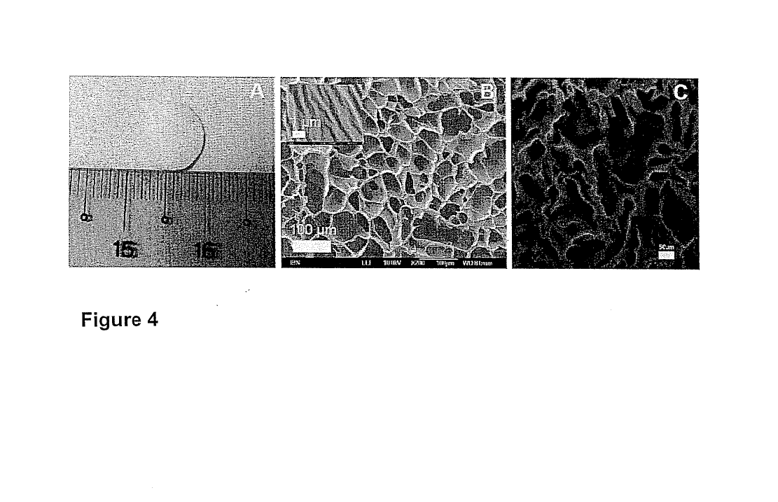

Surface morphology and porosity of the sponges were characterized using SEM. Image analysis of the sponge porosity revealed the average pore size to be between 70 to 110 μm; 50% of pores were 71-90 μm, 36% were 91-110 μm. Measurement of the elastic modulus of the sponges using atomic force microscopy revealed an average modulus of 30 kPa. This modulus was found to be slightly higher compared to the previous non-cleavable sponge but still considered to be as soft matrix. The sponge also had considerably high water uptake (˜96%). High magnification images of the sponge surface revealed surface roughness in the range of 0.5 μm scale which was proven previously to help tethering the hepatocyte spheroids to the sponge (FIG. 4B). The dry sponge was soaked in 0.25 mg/mL propidium iodide solution to stain the sponge's macroporous structure. By laser confocal microscopy, we observed that the macroporosity was maintained as a hydrated macroporous network structure in an aqueous environment (FIG. 4C) in contrast to typical hydrogels that lose their porosity in an aqueous environment.

Conjugated Disulfide Bonds on the Side Chain of Hydroxypropyl Cellulose Induces Cleavability of Macroporous Cellulosic Sponge Rapidly at Physiological Condition

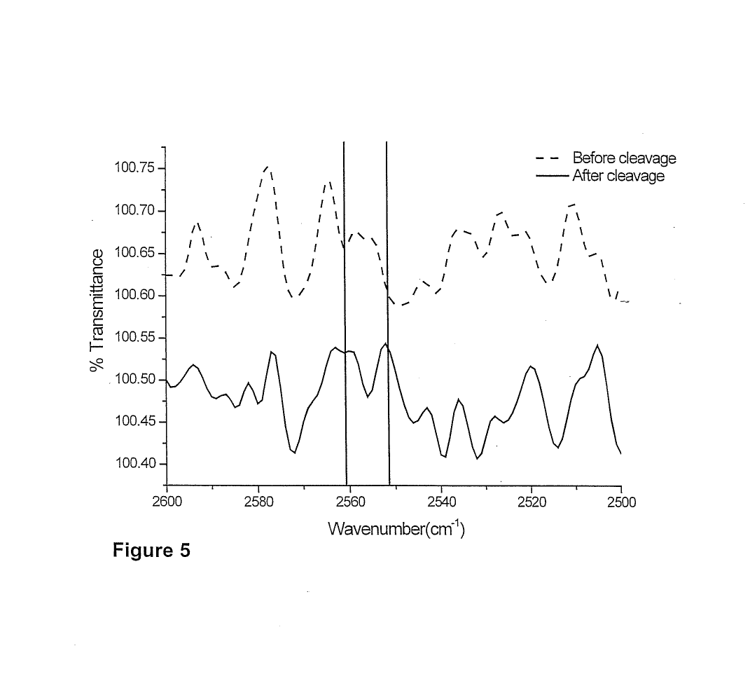

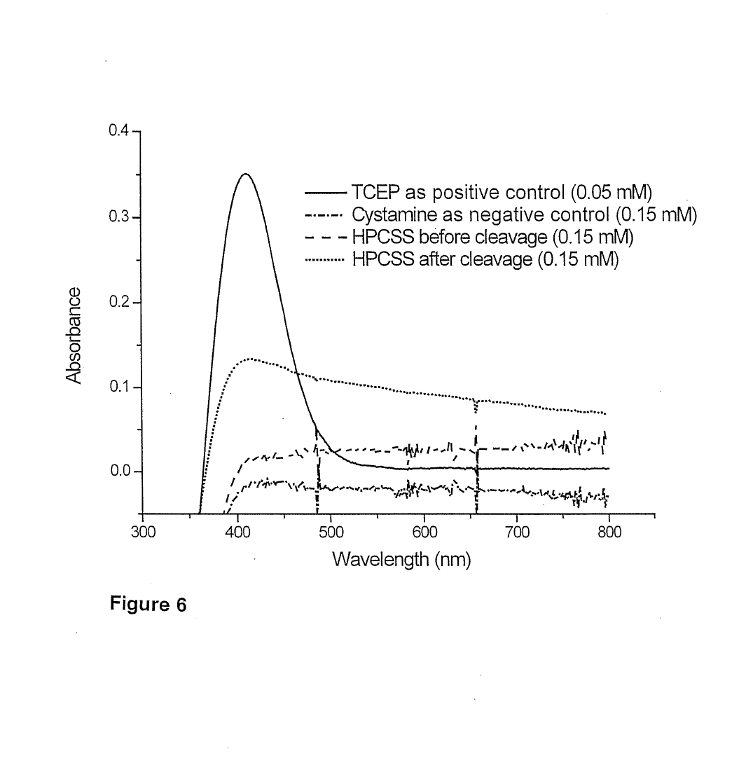

Upon successfully inserting disulfide bond onto hydroxypropyl cellulose side chain, the cleavability of the sponge in reductants was tested. Sponge samples before and after cleavage were compared and analyzed by FTIR. FTIR spectra showed the valley-like peak appearance at 2550 cm⁻¹, similarly as it was reported previously in another disulfide-containing hydrogel (FIG. 5) [21]. This peak corresponded to thiol group (—SH) formation from disulfide group (—S—S—) upon cleavage. The thiol group formed was further confirmed by Ellman's thiol analysis in terms of its ability to further cleave a known disulfide-containing dye 5,5′-dithiobis-(2-nitrobenzoic acid) (DTNB) to be UV-detectable compound. The cleaved sponge sample was found to be able to cleave DTNB at ˜⅓^{rd} strength of the positive control, TCEP (FIG. 6).

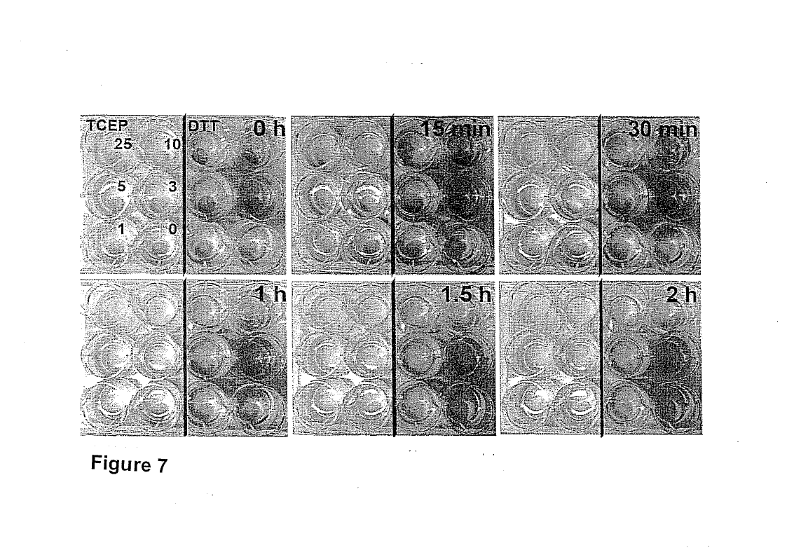

After confirming the formation of thiol groups from disulfide groups upon cleavage, the optimum cleavage condition was determined by incubating the sponge with serial concentrations of reductants (TCEP and DTT). DTT, a known potent reductant, was used as a positive comparator. Physical morphology changes of the sponge were monitored visually (FIG. 7). To ease the monitoring process, the sponges were pre-soaked overnight with 0.25 mg/mL propidium iodide. TCEP and DTT could cleave the sponge at similar strengths (Table 7). The optimum cleavage condition was determined by the time taken for cleavage as well as reductant concentration used. TCEP, a reductant without thiol group, had been favoured over thiol-based reductants due to its less cytotoxicity and less ability to penetrate cell membrane [27, 28]. Therefore, 10 mM TCEP was chosen as optimum condition for further sponge cleavage (˜30 minutes cleavage).

TABLE 7
Optimization of cellulosic sponge cleavage
using different concentrations of reductant

| TCEP | | DTT | |
|---|---|---|---|
| concentrations | | concentrations | |
| in William's E | | in William's E | |
| medium | Remarks | medium | Remarks |
| 25 mM | Sponge cleaved | 25 mM | Sponge cleaved |
| | within 30 minutes | | within 30 minutes |
| 10 mM | Sponge cleaved | 10 mM | Sponge cleaved |
| | within 30 minutes | | within 30 minutes |
| 5 mM | Sponge cleaved | 5 mM | Sponge cleaved |
| | within 1.5 hour | | within 1.5 hour |
| 3 mM | Sponge cleaved | 3 mM | Sponge cleaved |
| | within 2 hours | | within 2 hours |
| 1 mM | Sponge not | 1 mM | Sponge not |
| | cleaved | | cleaved |

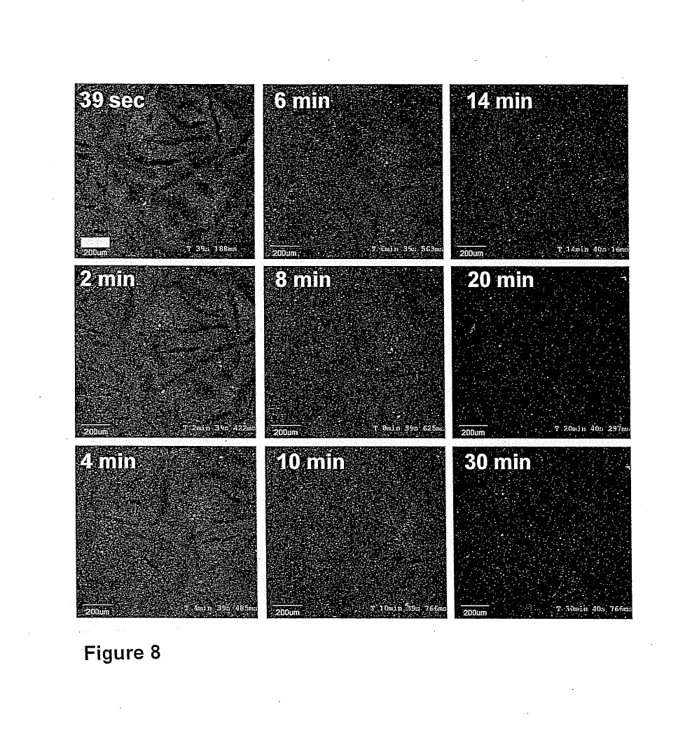

Sponge aqueous macroporous network disappearance was monitored dynamically by time lapse imaging in confocal microscopy. This salient feature of the cellulosic sponge was monitored within 30 minutes duration with image taken every 2 minutes time interval. As it is shown in FIG. 8, 6 minutes after adding 10 mM TCEP, the sponge macroporous networks had shrunken significantly. At 20 minutes, macroporosity had completely vanished with significant drop in the total signal intensity. This decrease corresponded to sponge thinning and dissolution during cleavage.

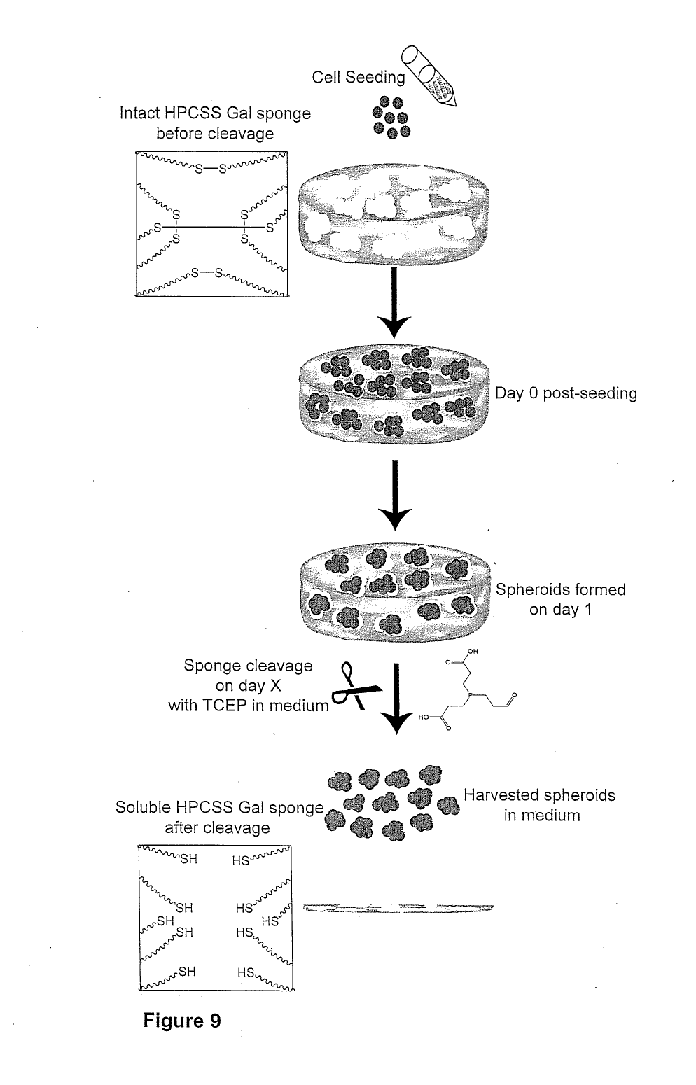

The intended applications of this cleavable cellulosic sponge were for 3D spheroids culture as well as its cleavability to retrieve the spheroids for further manipulations. This proposed mechanism is show in FIG. 9. During cleavage, disulfide bonds exchanged into thiol bonds, loosen up the crosslinked bond and made the sponge soluble. The cleavage should only occur on-demand upon adding the disulfide bond reductant.

Reductant Used to Cleave the Sponge is Relatively Non-Cytotoxic Even into Sensitive Cell Type (Primary Hepatocytes)

The cytotoxicity of TCEP was investigated by incubating different concentrations of TCEP with the associated time needed to completely cleave the sponge; 5 mM 1.5 hour, 10 mM 30 minutes and 25 mM 30 minutes. Since the application for this cleavable sponge was mainly for primary cells culture, primary rat hepatocyte was used in the cytotoxicity study.

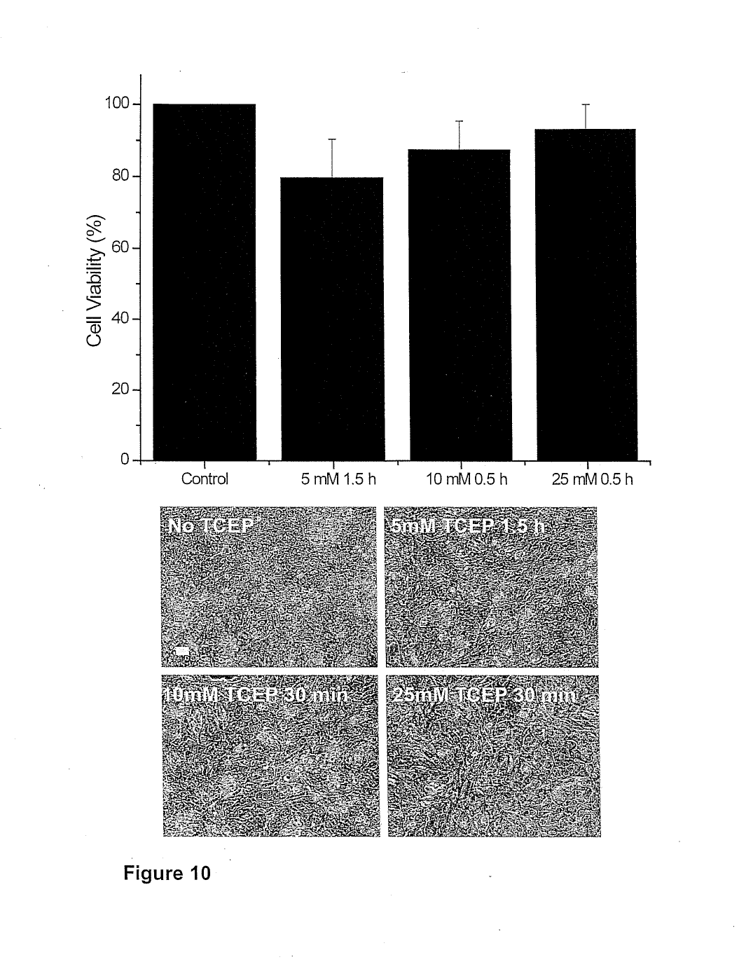

As it is shown in FIG. 10 upper panel, primary rat hepatocyte viability upon incubation of TCEP at configurations of 5 mM 1.5 hour, 10 mM 30 minutes and 25 mM 30 minutes were 79.75±10.71%, 87.42±8.03% and 93.06±6.96%, respectively. Phase contrast images of hepatocytes with and without TCEP incubation at the lower panel of FIG. 40 clearly show the maintenance of cuboidal cell shape and bile canaliculi-like structure at cell-cell contact. No significant cell damage was observed.

Characterization of the Hepatocyte Spheroids Cultured in Cellulosic Sponges

Primary Rat Hepatocytes Form Compact Hepatocyte Spheroids in the Cleavable Cellulosic Sponge within 24 Hours Post-Seeding

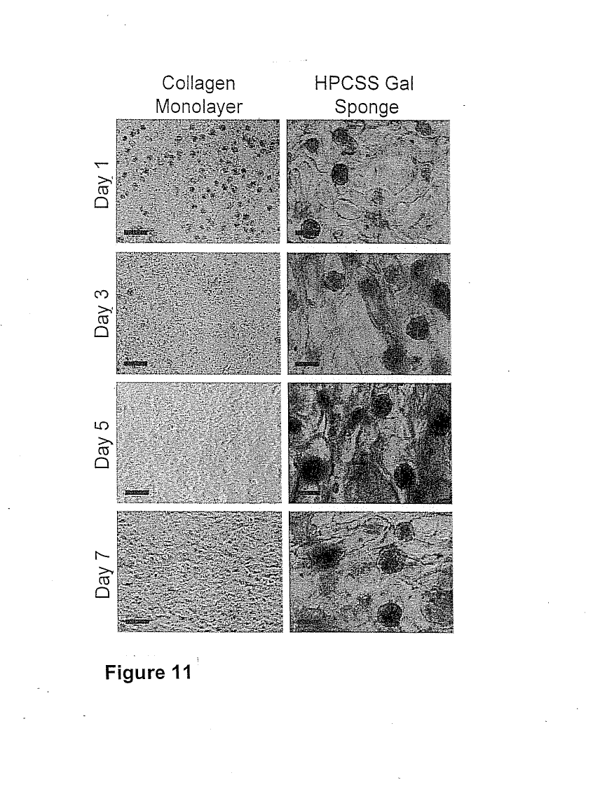

Similarly as it was shown previously in non-cleavable sponge (HA Gal sponge), in cleavable HPCSS Gal sponge, primary rat hepatocytes also immediately reorganized into 3D spheroids within 1 day of culture and remained stable in this configuration until at least day 7 (FIG. 11). Hepatocyte spheroids formed in the HPCSS Gal sponges were 88.77 μm in average diameter. The spheroids formed in HPCSS Gal sponges were constrained in the sponge macropores and thus did not easily detach as those previously shown on 2D PET Gal membranes [3].

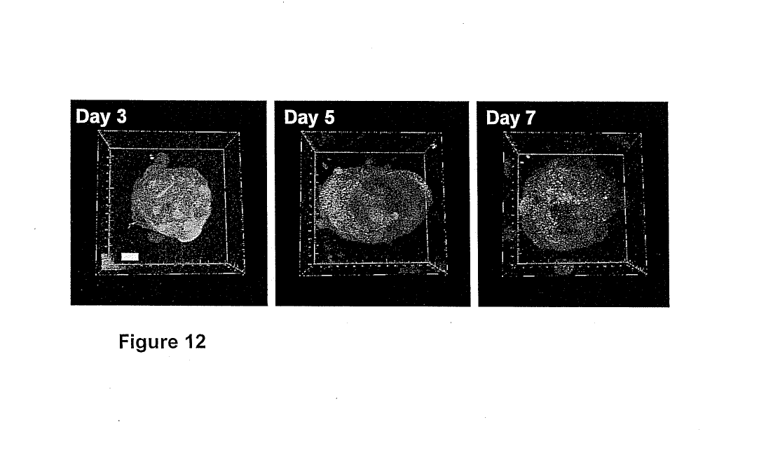

Hepatocyte spheroids viability, which was assessed by co-staining the cells with Cell-Tracker Green (CTG) and Propidium Iodide (PI), showed majority of green signals which revealed good viability maintenance at least 7 days in culture (FIG. 12). The CTG signals illustrated indistinguishable borders between single cells in the spheroids, which reflected the tightness of the cell-cell contacts.

Hepatocyte Spheroids Formed in Cleavable Cellulosic Sponges could be Retrieved by Cleaving the Sponge without Imposing Cytotoxicity

The effect of sponge cleavage to the characteristics of retrieved spheroid was investigated towards the downstream effects e.g. maintenance of cytochrome P450 genes, polarity marker, tight cell junction, spheroids compactness and bile excretory function. The maintenance of important rat cytochrome P450 genes (CYP1A2, CYP2B2 and CYP3A2) was analyzed based on the effect of TCEP incubation into short term and long term culture/post-replating. Hepatocyte spheroids cultured in non-cleavable sponge (HA Gal sponge) were used as a comparator.

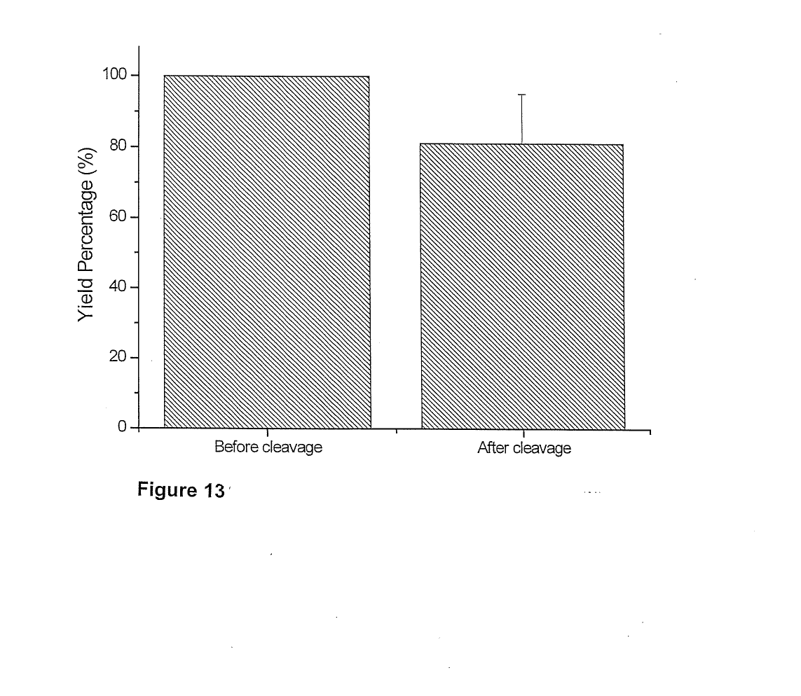

Rat hepatocyte spheroids incubation with 10 mM TCEP for 30 minutes did not show any significant detrimental effect to the 3 CYP genes (FIG. 13). There was slight drop in the fold expression changes of CYP genes upon cleaving the HPCSS Gal sponge to retrieve the spheroids (˜2 fold decrease). However, upon overnight replating these retrieved spheroids on poly-L-lysine coated dish, the spheroids rejuvenated the gene expressions comparable to the spheroids cultured in non-cleavable sponge.

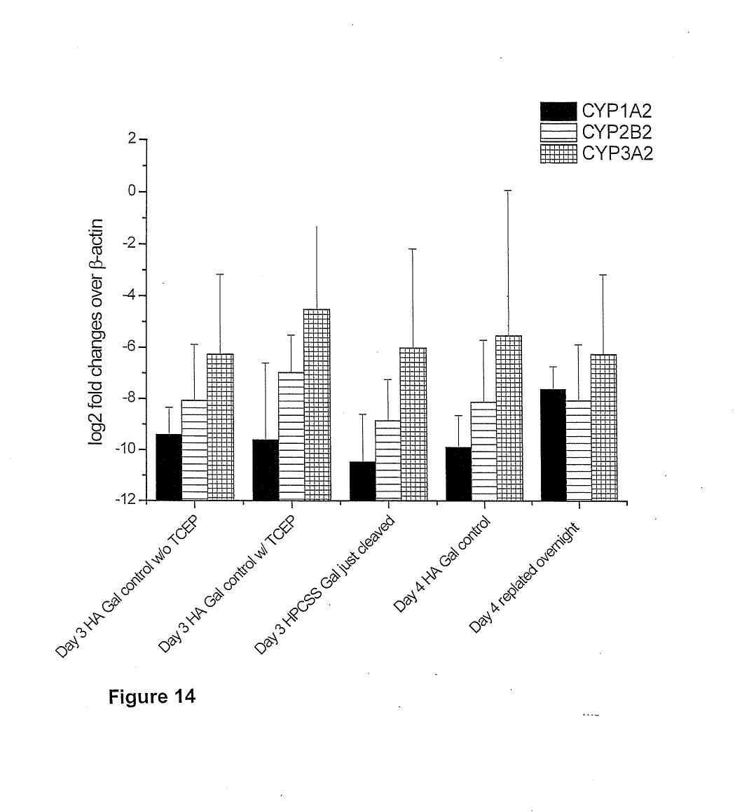

Immunofluorescence staining of F-actin, E-cadherin and MRP2 in the hepatocyte spheroids 72 hours post seeding retrieved from cleavable (HPCSS Gal) sponges and in non-cleavable (HA Gal) sponge and showed comparable localization of these markers (FIG. 14). As would be expected in non-spreading cells, F-actin staining revealed that the actin cytoskeleton had a predominant cortical localization in the spheroids and an absence of stress fibers. E-cadherin staining, a marker of cell-cell tight junctions demonstrated that cells in the hepatocyte spheroids have tight associations between neighbouring cells. MRP2 staining marked the apical domains of the polarized hepatocytes. In MRP2 staining image (FIG. 14 rightmost panel), the signals showed a comparable signal as observed in the intact hepatocyte spheroids in the non-cleavable sponge and retrieved spheroids from cleavable sponge.

Upon confirming the maintenance expression of MRP2 (apical domain marker as well as hepatocyte efflux transporter) in the retrieved spheroids, the excretory 131 function of these spheroids was studied by incubating the spheroids with fluorescein diacetate (FDA). Viable cells in the spheroids will cleave FDA into fluorosecein dye by intracellular esterases which then be excreted by MRP2 into the bile canaliculi. FDA staining of the retrieved hepatocyte spheroids showed dye accumulation in the bile canaliculi region between the two cells (FIG. 45). This excretion exhibited similarity to what had been observed in the intact hepatocyte spheroids in non-cleavable sponge.

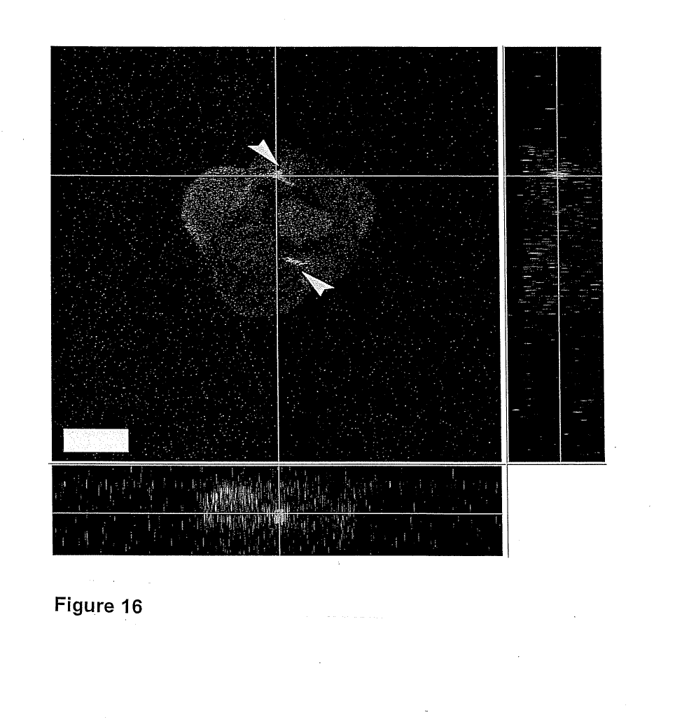

The maintenance of spheroids compact morphology upon retrieving was again confirmed by SEM. The retrieved hepatocyte spheroids still showed surface smoothness and disappearance of the cell-cell boundaries, compared to the spheroids cultured in non cleavable sponge (FIG. 16).

Retrieved Hepatocyte Spheroids Show Replatability and Easy Manipulation

The applicability of the retrieved spheroids obtained by cleaving the sponge was demonstrated by replating the spheroids on two different kinds of coated dishes; collagen and poly-L-lysine-coated dishes. These two kinds of coated dishes theoretically will either induce spheroids spreading or prevent spheroids spreading, respectively.

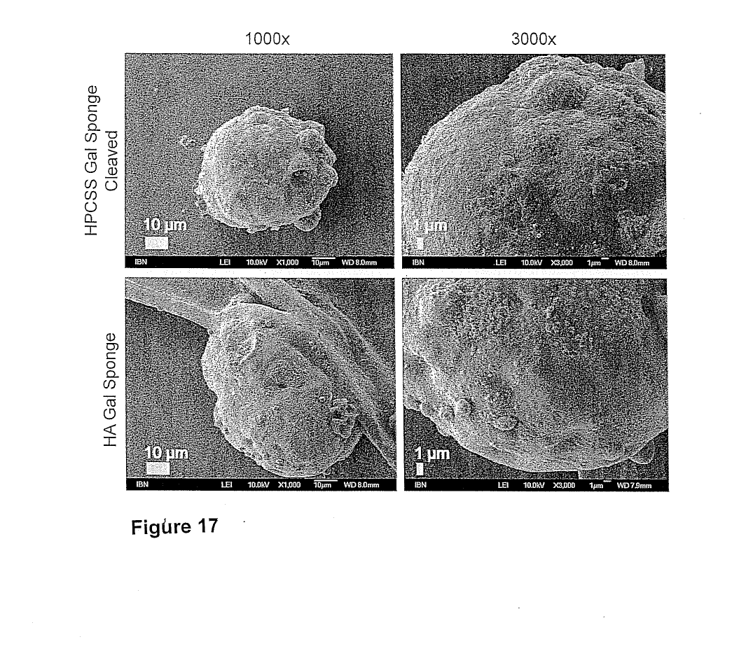

Upon spheroids replating on collagen coated dish, the spheroids settled on the bottom of the dish within 1 hour. 4 hours post-replating, the spreading pattern of the spheroids was clearly observed at the spheroids periphery (FIG. 17). And 16 hours later, the spheroids had exhibited an almost complete spreading. Some cells located at the centre of spheroids did not manage to spread due to cells hindrance beneath them. Cells which were spreading out of the spheroids showed the clear bile canaliculi-like structure at the cell-cell boundary, which was normally seen on hepatocyte collagen monolayer culture.

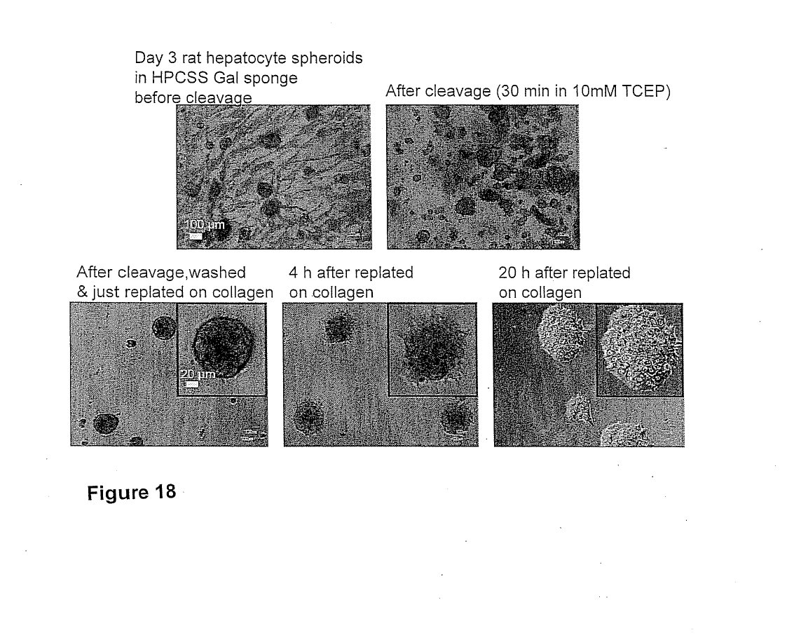

Instead of allowing the spheroids to spread, the spheroids compact morphology could be maintained for extended period by replating them on poly-L-lysine coated dish. Positively charged polymer such as poly-L-lysine has been known to anchor hepatocyte spheroids but prevent spheroid spreading [29]. When the hepatocyte spheroids were retrieved and replated on poly-L-lysine coated dish, the spheroids settled at the bottom of the dish and remained as intact round spheroids (FIG. 18, upper panel). The spheroids also showed good cell viability upon replating (FIG. 18, lower panel).

Cleavable Sponge Exhibits Utility for 3D Spheroids Culture and Retrieval of Cancer and Stem Cells

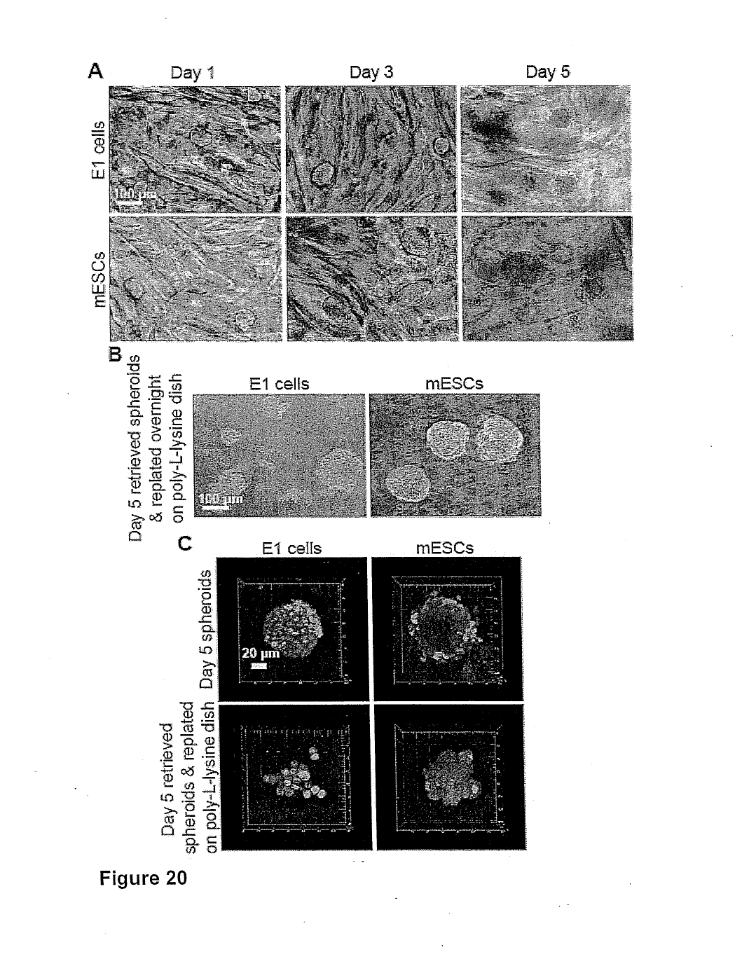

Not limited to only hepatocyte-based applications, our cleavable cellulosic sponge also shows similar utility for 3D cancer cell and stem cell cultures (FIG. 20). Both E1 (metastatic derivative human colorectal cancer cells) and mESCs cells seeded into cleavable HPCSS sponges reorganized into compact spheroids within 24 hours post-seeding. The spheroids remained compact at least up to 5 days of culture, were easily retrieved through sponge cleavage and maintained good cell viability during the culture and post-retrieval.

Discussion

We have conjugated disulfide bonds onto side chain group of hydroxypropyl cellulose to create cleavable hydrogel-based sponge. This strategy has shown successful rapid and easy cleavage of cellulosic sponge without hindering the ability of hydroxypropyl cellulose to be crosslinked by y irradiation and formed macroporous sponge. The sponge is fabricated without chemical cross-linkers, yet cross-linked through stable chemical bonds. Made of a water soluble precursor, our cleavable cellulosic hydrogel sponges are very hydrophilic thus acting as a non-adhesive matrix and preventing cell spreading, which is important for maintenance of the mature hepatocyte phenotype [30].

The sponge was also easy to be galactosylated for specific hepatocyte culture application. Unlike other hydrogels, the macroporous networks in our cellulosic sponge support the in situ formation and maintenance of polarized hepatocyte spheroids. The cellulosic sponge, which acts as a hepatocyte substratum anchor, did not prevent cell aggregation, as would normally happen in cell culture hydrogel with excessive extracellular matrix presentation [34]. The galactose presented chemical cues to the hepatocytes to reorganize into 3D spheroids within 1 day post-seeding, while the macroporous structure constrained them physically. Since the galactose ligand only interacts weakly with ASGPR receptors in the hepatocyte cell membrane [35], it is the combination of the physical and chemical cues in the sponge which is important in establishing stable constrained hepatocyte spheroids.

The cleavable sponge still exhibited soft hydrogel mechanical stiffness characteristic (E≈30 kPa) with excellent water uptake (>95%) despite the alteration of side chain chemistry with disulfide bond. Excellent sponge water uptake combined with structurally desirable characteristics make this a suitable scaffold for 3-Dimensional growth of cell cultures. We thus ensured short exposure of the cells to reductant solution. manipulate the spheroids easily. Overall, our cleavable cellulosic sponge provided the facile hydrogel-based sponge platform to culture hepatocytes, cancer and stem cells as 3D spheroids with the ease of spheroids retrieval through non-cytotoxic sponge cleavage.

### CONCLUSION

We have synthesized and fabricated a cleavable macroporous cellulosic hydrogel sponge conjugated with galactose as a platform to culture primary rat hepatocytes as 3D spheroids with the ability to retrieve the spheroids at physiological condition. Hepatocyte spheroids retrieval was performed through rapid non-cytotoxic sponge cleavage. The soft macroporous structure of cleavable cellulosic sponge conjugated with galactose facilitated the formation of hepatocyte spheroids by presenting both the mechanical cues (via matrix rigidity) and chemical cues for the hepatocytes to reorganise into 3D spheroids within 24 hours post-seeding. The constrained hepatocyte spheroids maintained cell viability for at least a week of culture. Upon spheroids retrieval through sponge cleavage, polarized hepatocyte phenotypes were well maintained; drug metabolizing enzymes (CYP1A2, CYP2B2 and CYP3A2), polarity marker (cortical F-actin), tight cell-cell junction, apical hepatocyte domain marker (MRP2), biliary excretory function, spheroid compact morphology and cell viability. The living retrieved spheroids were replatable on both collagen coated and poly-L-lysine coated dishes for further use. The utility of our cleavable sponge also extends to 3D spheroids culture of cancer and stem cells for 3D engineered tumor model and stem cell differentiation platforms. As many of tissue engineering-related researches require temporary synthetic scaffold matrix such as stem cell differentiation and in vitro organoid and thick tissue culture, this technology offers useful platform to culture cells as 3D spheroids construct with easy, rapid and physiological removal of bulk synthetic sponge.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### REFERENCES

[1] Chien H W, Tsai W B, Jiang S. Direct cell encapsulation in biodegradable and functionalizable carboxybetaine hydrogels. Biomaterials. 2012; 33:5706-12.
[2] Yue Z, Wen F, Gao S, Ang M Y, Pallathadka P K, Liu L, et al. Preparation of three-dimensional interconnected macroporous cellulosic hydrogels for soft tissue engineering. Biomaterials. 2010; 31:8141-52.
[3] Nugraha B, Hong X, Mo X, Tan L, Zhang W, Chan P M, et al. Galactosylated cellulosic sponge for multi-well drug safety testing. Biomaterials. 2011; 32:6982-94.
[4] Gu H, Yue Z, Leong W S, Nugraha B, Tan L P. Control of in vitro neural differentiation of mesenchymal stem cells in 3D macroporous, cellulosic hydrogels. Regenerative medicine. 2010; 5:245-53.
[5] Habib Onsori M R Z, Mostafa Motallebi, Nosratollah Zarghami. Identification of over producer strain of endo-b-1,4-glucanase in Aspergillus Species: Characterization of crude carboxymethyl cellulase. African Journal of Biotechnology 2005; 4:26-30.
[6] Zilberman M, Nelson K D, Eberhart R C. Mechanical properties and in vitro degradation of bioresorbable fibers and expandable fiber-based stents. Journal of biomedical materials research Part B, Applied biomaterials. 2005; 74:792-9.
[7] Dispinar T, Van Camp W, De Cock L J, De Geest B G, Du Prez F E. Redox-responsive degradable PEG cryogels as potential cell scaffolds in tissue engineering. Macromol Biosci. 2012; 12:383-94.
[8] Kloxin A M, Kasko A M, Salinas C N, Anseth K S. Photodegradable hydrogels for dynamic tuning of physical and chemical properties. Science. 2009; 324:59-63.
[9] Meng F, Hennink W E, Zhong Z. Reduction-sensitive polymers and bioconjugates for biomedical applications. Biomaterials. 2009; 30:2180-98.
[10] Hamid Z A, Blencowe A, Ozcelik B, Palmer J A, Stevens G W, Abberton K M, et al. Epoxy-amine synthesised hydrogel scaffolds for soft-tissue engineering. Biomaterials. 2010; 31:6454-67.
[11] Raines CPaRT. Adjacent cysteine residues as a redox switch. Protein Eng 2001; 14:939-42.
[12] Liu Y, Zheng Shu X, Prestwich G D. Biocompatibility and stability of disulfide-crosslinked hyaluronan films. Biomaterials. 2005; 26:4737-46.
[13] Hisano N, Morikawa N, Iwata H, Ikada Y. Entrapment of islets into reversible disulfide hydrogels. Journal of biomedical materials research. 1998; 40:115-23.
[14] Bromberg L, Temchenko M, Alakhov V, Hatton T A. Kinetics of swelling of polyether-modified poly(acrylic acid) microgels with permanent and degradable cross-links. Langmuir: the ACS journal of surfaces and colloids. 2005; 21:1590-8.
[15] Plunkett K N, Berkowski K L, Moore J S. Chymotrypsin responsive hydrogel: application of a disulfide exchange protocol for the preparation of methacrylamide containing peptides. Biomacromolecules. 2005; 6:632-7.
[16] Yoshida H, Klinkhammer K, Matsusaki M, Moller M, Klee D, Akashi M. Disulfide-crosslinked electrospun poly(gamma-glutamic acid) nonwovens as reduction-responsive scaffolds. Macromol Biosci. 2009; 9:568-74.
[17] Kakizawa Y, Harada A, Kataoka K. Glutathione-sensitive stabilization of block copolymer micelles composed of antisense DNA and thiolated poly(ethylene glycol)-block-poly(L-lysine): a potential carrier for systemic delivery of antisense DNA. Biomacromolecules. 2001; 2:491-7.
[18] Lees W J, and Whitesides, G. M. Equilibrium Constants for Thiol-Disulfide Interchange Reactions. The Journal of organic chemistry. 1993; 58:642-7.
[19] Shu X Z, Liu Y, Palumbo F, Prestwich G D. Disulfide-crosslinked hyaluronan-gelatin hydrogel films: a covalent mimic of the extracellular matrix for in vitro cell growth. Biomaterials. 2003; 24:3825-34.
[20] John A. Burns J C B, John Moran, and George M. Whitesides. Selective Reduction of Disulfides by Tris(2-carboxyethyl)phosphine The Journal of organic chemistry. 1991; 56:2648-50.
[21] Matsusaki M, Yoshida H, Akashi M. The construction of 3D-engineered tissues composed of cells and extracellular matrices by hydrogel template approach. Biomaterials. 2007; 28:2729-37.
[22] http://www.qcbio.com/uploaddrivers/233153-000.pdf.
[23] Han J C, Han G Y. A procedure for quantitative determination of tris(2-carboxyethyl)phosphine, an odorless reducing agent more stable and effective than dithiothreitol. Analytical biochemistry. 1994; 220:5-10.
[24] http://www.piercenet.com/instructions/2161434.pdf.
[25] Ren X L, Yu Jin; Han, Ho Jae; Kim, In S. Effect of tris-(2-chloroethyl)-phosphate (TCEP) at environmental concentration on the levels of cell cycle regulatory protein expression in primary cultured rabbit renal proximal tubule cells. Chemosphere. 2008; 74:84-8.
[26] Seglen P O. Preparation of isolated rat liver cells. Methods Cell Biol. 1976; 13:29-83.
[27] TCEP—a reagent of choice in both Proteomics and nucleic acid chemistry. http://www.polyorganix.com/productsubcat.php?category=VENFUCBwcm9kdWNOcw==&catid=17
[28] Shi X, Garcia G E, Neill R J, Gordon R K. TCEP treatment reduces proteolytic activity of BoNT/B in human neuronal SHSY-5Y cells. Journal of cellular biochemistry. 2009; 107:1021-30.
[29] Hamamoto R, Yamada K, Kamihira M, lijima S. Differentiation and proliferation of primary rat hepatocytes cultured as spheroids. Journal of biochemistry. 1998; 124:972-9.
[30] Wells R G. The role of matrix stiffness in regulating cell behavior. Hepatology. 2008; 47:1394-400.
[31] Hsiao C C, Wu J R, Wu F J, Ko W J, Remmel R P, Hu W S. Receding cytochrome P450 activity in disassembling hepatocyte spheroids. Tissue engineering. 1999; 5:207-21.
[32] Ranucci C S, Kumar A, Batra S P, Moghe P V. Control of hepatocyte function on collagen foams: sizing matrix pores toward selective induction of 2-D and 3-D cellular morphogenesis. Biomaterials. 2000; 21:783-93.
[33] Kim M, Lee J Y, Jones C N, Revzin A, Tae G. Heparin-based hydrogel as a matrix for encapsulation and cultivation of primary hepatocytes. Biomaterials. 2010; 31:3596-603.
[34] Green J E, Waters S L, Shakesheff K M, Byrne H M. A mathematical model of liver cell aggregation in vitro. Bull Math Biol. 2009; 71:906-30.
[35] Du Y, Chia S M, Han R, Chang S, Tang H, Yu H. 3D hepatocyte monolayer on hybrid RGD/galactose substratum. Biomaterials. 2006; 27:5669-80.

1. A method of making a scaffold for 3 dimensional cell culture comprising the steps of conjugating a reducible disulfide bond onto a hydroxyl group at the side chain of a hydroxypropyl cellulose; forming a matrix of hydroxypropyl cellulose having the reducible disulfide bond conjugated onto the hydroxyl group such that the reducible disulfide bond exists adjacent to a double bond for crosslinking the matrix of the hydroxypropyl cellulose.

2. The method of claim 1 wherein the matrix comprises a macroporous network.

3. The method of claim 1 further comprising the step of conjugating a ligand onto the matrix.

4. The method of claim 3 wherein the ligand comprises galactose.

5. The method of claim 3 wherein the ligand comprises a primary amine.

6. The method of claim 1 wherein the reducible disulfide bond comprises a dithiodipropionic acid.

7. The method of claim 1 wherein the double bond crosslinking the matrix of hydroxypropyl cellulose is crosslinked by gamma radiation.

8. The method of claim 1 wherein the reducible disulfide bond is reducible by a reductant.

9. The method of claim 8 wherein the reductant is tris(2-carboxyethyl)phosphine (TCEP).

10. A scaffold for 3 dimensional cell culture comprising a reducible disulfide bond conjugated onto a hydroxyl group at the side chain of a hydroxypropyl cellulose adjacent to a double bond for crosslinking a matrix of the hydroxypropyl cellulose.

11. The scaffold of claim 10 wherein the matrix comprises a macroporous network.

12. The scaffold of claim 10 further comprising a ligand conjugatable onto the matrix.

13. The scaffold of claim 12 wherein the ligand comprises galactose.

14. The scaffold of claim 12 wherein the ligand comprises a primary amine.

15. The scaffold of claim 10 wherein the reducible disulfide bond comprises a dithiodipropionic acid.

16. The scaffold of claim 10 wherein the reducible disulfide bond is reducible by a reductant.

17. The scaffold of claim 16 wherein the reductant is tris(2-carboxyethyl)phosphine (TCEP).

18. A system of culturing 3 dimensional cell spheroids comprising, seeding cells on a scaffold of claim 10; and incubating the cells in a cell growth medium allowing the cells to grow into a spheroid.

19. The system of claim 18 further comprising cleaving the disulfide bond with a reductant to retrieve the spheroid.

20. The system of claim 19 wherein the reductant is tris(2-carboxyethyl)phosphine (TCEP).

21. The system of claim 18 wherein the cells are hepatocytes.

22. The system of claim 18 wherein the cells are metastatic cancer cells.

23. The system of claim 18 wherein the cells are pluripotent cells.


SEQUENCE LISTING (6 sequences)

SEQ ID NO: 1 - DNA, 20 bp, Artificial Sequence
  Synthetic Oligonucleotide
cacggctttctgacagaccc

SEQ ID NO: 2 - DNA, 20 bp, Artificial Sequence
  Synthetic Oligonucleotide
ccaagccgaagagcatcacc

SEQ ID NO: 3 - DNA, 20 bp, Artificial Sequence
  Synthetic Oligonucleotide
accggctaccaacccttgat

SEQ ID NO: 4 - DNA, 24 bp, Artificial Sequence
  Synthetic Oligonucleotide
tgtgtggtactccaatagggacaa

SEQ ID NO: 5 - DNA, 21 bp, Artificial Sequence
  Synthetic Oligonucleotide
tgggacccgcacacatggact

SEQ ID NO: 6 - DNA, 22 bp, Artificial Sequence
  Synthetic Oligonucleotide
tccgtgatggcaaacagaggca